(19) United States
(12) Patent Application Publication
(10) Pub. No.: US 2024/0382351 A1
(43) Pub. Date: Nov. 21, 2024

(54) DIAPER HAVING A DEPLOYABLE CONTAINMENT BAG

(71) Applicant: Elias Hilal, Davie, FL (US)

(72) Inventor: Elias Hilal, Davie, FL (US)

(21) Appl. No.: 18/665,910

(22) Filed: May 16, 2024

Related U.S. Application Data
(60) Provisional application No. 63/466,952, filed on May 16, 2023.

(51) Int. Cl. A61F 13/495

(52) U.S. Cl. CPC A61F 13/495

(57) ABSTRACT

A diaper assembly having a deployable containment bag, with the assembly including a diaper member and a containment bag which is sized to be able to enclose the diaper member, with the containment bag having removably stored in the diaper member. The diaper member is configured to allow the containment bag to be both stored therein and selectively removed therefrom without affecting the diaper member's ability to absorb or contain the waste of the wearer. Once the containment bag is removed from the diaper member, it can be opened and thereafter allow the diaper member to be placed inside it. The containment bag can then be sealed closed. In this regard, the containment be that provides for an integrated containment system which enables a wet and/or soiled diaper to be sealed in an airtight space to make disposal more convenient and sanitary.

### CROSS REFERENCE TO RELATED APPLICATIONS

This application claims the benefit of and incorporates by reference co-pending U.S. provisional patent application Ser. No. 63/466,952 filed May 16, 2023.

### BACKGROUND OF THE INVENTION

### Field of the Invention

This invention relates generally to diapers and, more particularly, to a diaper having an integrated containment article which can be selectively deployed and thereafter employed to enclose the diaper.

### Description of the Prior Art

The use and design of diapers as a form of underwear which is suitable for absorbing or containing waste and thereby preventing the soiling of clothing or other environmental items is well known. While diapers are most commonly worn by infants and young people who have not yet learned to use the toilet each time they urinate and/or defecate, their use by adults in some circumstances and by the elderly is also well established. In this regard, a key advantage of a diaper, whether it be an infant diaper, training pants, or an adult incontinence product, is that they allow the wearer to urinate and/or defecate where they are at a given moment and not have to find a toilet or other suitable area in order to do so.

That said, a well-known limitation of a diaper is that when it becomes wet and/or soiled with waste from the wearer, it must be taken off the wearer sooner rather than later. Indeed, if a wet and/or soiled diaper is not removed from the wearer soon enough, it can cause skin irritation or other problems in the area covered by the diaper. But once removed, it can be difficult to find a suitable place to dispose of a wet and/or soiled diaper. This is because a wet and/or soiled diaper will often emanate a strong odor from the waste therein, and in social environments, it is frowned upon to leave something giving off the strong odor of such waste in many places that otherwise are appropriate for trash (even trash receptacles if they are uncovered).

Thus, there remains a need for a diaper which has deployable containment bag integrated therewith which can be selectively employed to enclose a wet and/or soiled diaper. It would additionally be desirable for the containment bag of the diaper having a deployable containment bag to be deployable from a sealed pouch which is built into the diaper such that it remains flush with the exterior surface of the diaper. It would furthermore be desirable for the containment bag of the diaper having a deployable containment bag to have an air freshening and/or odor reducing element integral therewith.

### SUMMARY OF THE INVENTION

The present disclosure provides for a diaper assembly with a deployable containment bag, comprising: a diaper member configured to be worn by a user and at least one of absorb and contain urine and feces to prevent soiling of clothing that is also being worn by the user, wherein the diaper member includes an exterior surface; a containment bag defined by a non-rigid container having an open end and an interior portion, wherein when the containment bag is in a deployed configuration, the containment bag and open end are sized to allow the diaper member to pass through the open end and be contained in the interior portion; a first containment slot integral with the diaper member; a first slot cover integral with the diaper member and configured to be removably positioned over the first containment slot; and wherein when the containment bag is in a storage configuration and the first slot cover positioned over the first containment slot, the containment bag is sized to be enclosed in the first containment slot beneath the first slot cover.

It is an object of the present disclosure to provide a diaper which has deployable containment bag integrated therewith which can be selectively employed to enclose a wet and/or soiled diaper.

It is an additional object of the present disclosure to provide a diaper which has deployable containment bag that is deployable from a sealed pouch which is built into the diaper such that it remains flush with the exterior surface of the diaper.

It is an additional object of the present disclosure to provide a diaper which has deployable containment bag which has an air freshening and/or odor reducing element integral therewith.

### BRIEF DESCRIPTION OF THE DRAWINGS

FIG. 1 is a front elevational view of a diaper having a deployable containment bag built in accordance with an embodiment of the present invention.

FIG. 2 is a rear elevational view of a diaper having a deployable containment bag built in accordance with an embodiment of the present invention.

FIG. 3 is a rear elevational view of the diaper member of a diaper having a deployable containment bag built in accordance with an embodiment of the present invention having the slot cover removed.

FIG. 4 is a rear elevational view of a diaper having a deployable containment bag built in accordance with an embodiment of the present invention in a deployed configuration.

FIG. 5 is a front elevational view of a deployable containment bag built in accordance with an embodiment of the present invention in a deployed configuration.

FIG. 6 is a front elevational view of a deployable containment bag built in accordance with an embodiment of the present invention in a storage configuration.

FIG. 7 is a front elevational view of a diaper having a deployable containment bag built in accordance with an alternate embodiment of the present invention.

### DETAILED DESCRIPTION OF THE INVENTION

Described herein is a diaper having a deployable containment bag having a diaper member having removably stored in it a containment bag which is sized to be able to enclose the diaper. The diaper member is configured to allow the containment bag to be stored therein and be selectively removed therefrom without affecting the diaper member's ability to absorb or contain the waste of the wearer. Once the containment bag is removed from the diaper member, it can be opened and thereafter allow the diaper member to be placed inside it. The containment bag can then be sealed closed. In this regard, the containment be that provides for an integrated containment system which enables a wet and/or soiled diaper to be sealed in an airtight space to make disposal more convenient and sanitary.

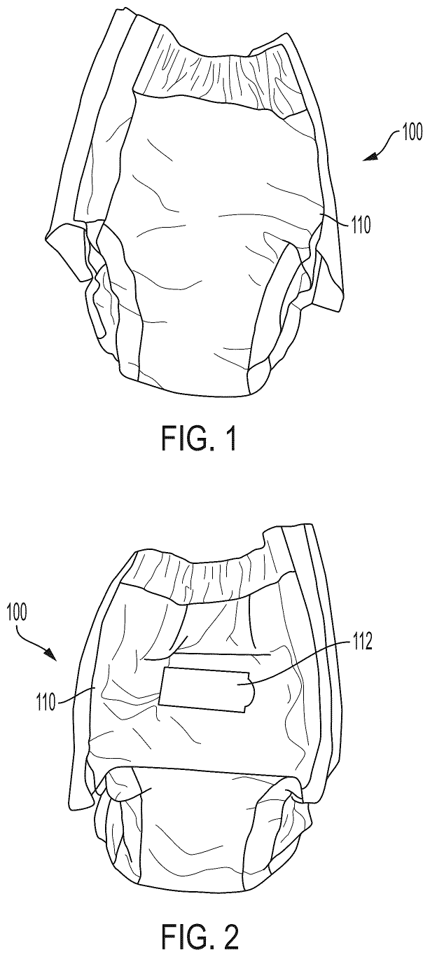

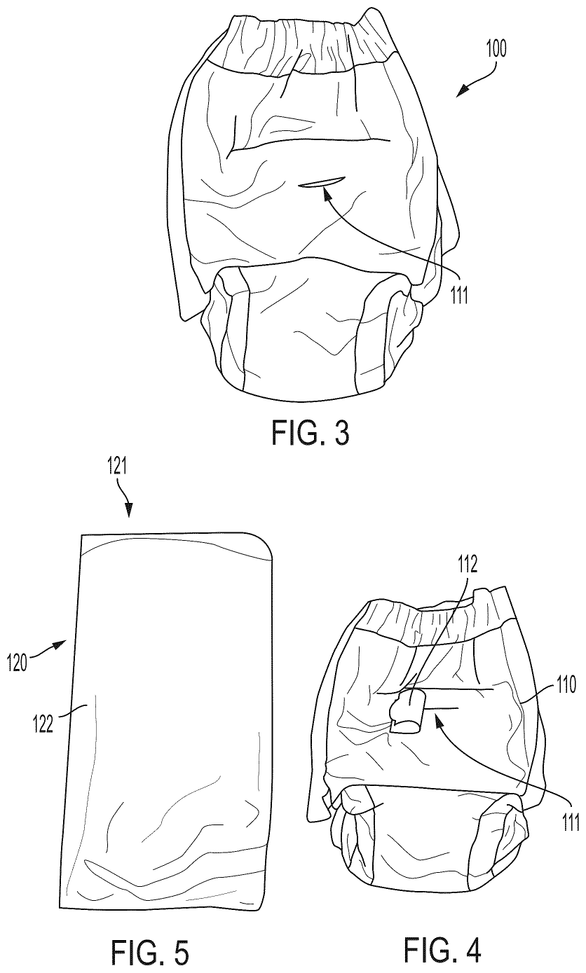

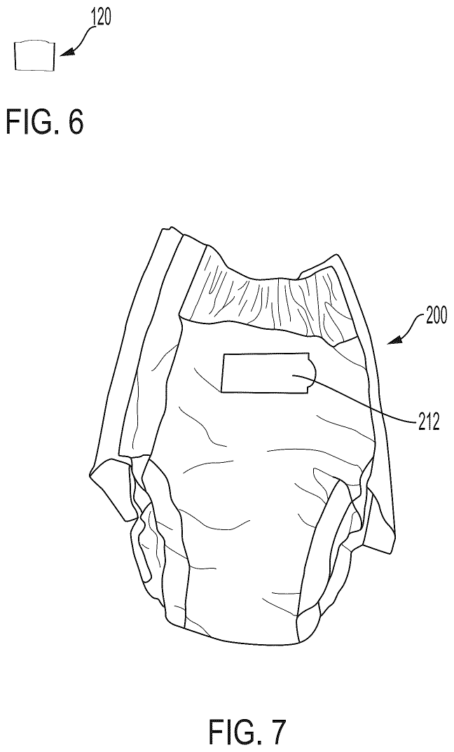

Referring now to the drawings and, in particular, FIGS. 1, 2, 3, 4, 5 and 6, a diaper having a deployable containment bag 100 is shown having a diaper member 110 with a containment slot 111 and a removable slot cover 112 as well as a containment bag 120. The diaper member 110 in the illustrated embodiment is defined by a conventional disposable training pants style diaper adapted to absorb and/or contain waste products such as human urine or feces to prevent soiling of clothing or other environmental items as conventional diapers. It is contemplated, however, that the diaper member 110 may alternatively be defined an infant diaper or an adult diaper. In this regard, it is appreciated that the diaper member 110 may be constructed in a plurality of sizes, and may employ any one of a variety of established structural designs.

The containment slot 111 defines a depression in the exterior surface of the diaper member 110. In the illustrated embodiment, the containment slot 111 is elongated and extends laterally across an upper portion on the rear exterior of the diaper member 110. It is contemplated that this positioning puts the slot in a location which is less directly responsible for absorbing and containing waste while being far enough away from the elastic portions of the waist area of the diaper to not affect the proper operation of the elastic areas. That said, because the relatively small width and depth of the containment slot 111, and that the exterior surface of the diaper member 110 remains continuous and unbroken in and around the containment slot 111, the diaper member 110 is structured such that the location of the containment slot 111 retains essentially the same absorptive and containment of waste properties as the areas of the diaper member 110 adjacent to the containment slot 111.

While the containment slot 111 in the illustrated embodiment is shown as elongated, it is contemplated that in alternate embodiments, it may be formed in alternate configurations such as rectangular or circular.

The removable slot cover 112 is defined by a releasably attached sheet which is sized to be positioned over and completely cover the containment slot 111 and thereby enclose the depression which defines the containment slot 111. In the illustrated embodiment, the slot cover 112 is constructed of a flexible piece of plastic that is coated on one side with an adhesive so as to allow it to releasably attach to the diaper member 110. Embodiments of the slot cover 112, however, may employ alternative mechanisms for releasably attaching to the diaper member 110, such as a hook and loop fastener material.

The containment bag 120 is defined by a non-rigid container having an open end 121 and closed sides 122, as well as an interior portion which is surrounded by the open end 121 and closed sides 122. The containment bag 120 is sized such that once it is deployed and opened, it can receive and hold the diaper member 110 in the interior portion. The containment bag 120 may be constructed of polyethylene. The open end 121 is sized such that it can both allow a diaper to pass into the interior portion and also be closed shut by tying a knot using the portion of the containment bag 120 adjacent to the rim around the open end 121 to cause the interior portion to become an airtight enclosure. It is contemplated that the containment bag 120 may include a coating of an air freshening and/or odor reducing substance on its exterior and/or interior surface.

Embodiments of the containment bag 120 may alternatively include interlocking plastic strips or an adhesive surface adjacent to the portion of the containment bag 120 adjacent to the rim around the open end 121 to allow for it to be sealed.

In some embodiments, the slot cover 112 may be integral with the containment bag 120 such that removing the slot cover 112 automatically causes the containment bag 120 to deploy.

A fully assembled diaper having a deployable containment bag 100 may have a single containment bag 120 folded up into a storage configuration and positioned in the containment slot 111, with the slot cover 112 positioned over the containment slot 111 to enclose the containment slot 111 with the containment bag 120 positioned therein. Then, if it is desired to deploy the containment bag 120, the slot cover 112 may be pulled off of the containment slot 111 and the containment bag 120 can be removed from the containment slot 111.

Referring now to FIG. 7, embodiments of the diaper having a deployable containment bag 200 may additionally include a secondary slot in its front surface. In such embodiments, the secondary slot may include a removable secondary cover 212 covering the secondary slot. The secondary slot and secondary cover 212 may be constructed in the same manner as the containment slot and slot cover described above, and may contain a deployable wet wipe to allow the cleaning of the skin during a diaper change.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A diaper assembly with a deployable containment bag, comprising:
a diaper member configured to be worn by a user and at least one of absorb and contain urine and feces to prevent soiling of clothing that is also being worn by the user, wherein the diaper member includes an exterior surface;
a containment bag defined by a non-rigid container having an open end and an interior portion, wherein when the containment bag is in a deployed configuration, the containment bag and open end are sized to allow the diaper member to pass through the open end and be contained in the interior portion;
a first containment slot integral with the diaper member;
a first slot cover integral with the diaper member and configured to be removably positioned over the first containment slot; and
wherein when the containment bag is in a storage configuration and the first slot cover positioned over the first containment slot, the containment bag is sized to be enclosed in the first containment slot beneath the first slot cover.

2. The diaper assembly with a deployable containment bag of claim 1, wherein the exterior surface of the diaper member is continuous in and around the first containment slot.

3. The diaper assembly with a deployable containment bag of claim 2, wherein the first containment slot is defined by a depression in the exterior surface of the diaper member.

4. The diaper assembly with a deployable containment bag of claim 1, wherein first containment slot extends laterally across an upper portion on a rear exterior of the diaper member.

5. The diaper assembly with a deployable containment bag of claim 1, wherein first containment slot is elongated and extends laterally across an upper portion on a rear exterior of the diaper member.

6. The diaper assembly with a deployable containment bag of claim 5, additionally comprising a second containment slot integral with the diaper member at an upper portion on a front exterior of the diaper member and a second slot cover integral with the diaper member and configured to be removably positioned over the second containment slot.

7. The diaper assembly with a deployable containment bag of claim 1, wherein the first slot cover is configured to be removably attached to the exterior surface of the diaper member adjacent to the first containment slot and when attached to the exterior surface of the diaper member adjacent to the first containment slot, is sized to extend over the first containment slot.

8. The diaper assembly with a deployable containment bag of claim 7, wherein the first slot cover is configured to be removably attached to the exterior surface of the diaper member with an adhesive coating.

9. The diaper assembly with a deployable containment bag of claim 1, wherein the containment bag integral with the first slot cover such that when the first slot cover is removed from a position over the first containment slot, the containment bag is automatically removed from the first containment slot.

10. The diaper assembly with a deployable containment bag of claim 1, wherein when the containment bag is in the deployed configuration, the containment bag is structured such that the open end can be sealed and cause the interior portion becoming an airtight enclosure.

11. A diaper assembly with a deployable containment bag, comprising:
a diaper member configured to be worn by a user and at least one of absorb and contain urine and feces to prevent soiling of clothing that is also being worn by the user, wherein the diaper member includes an exterior surface;
a containment bag defined by a non-rigid container having an open end and an interior portion, wherein when the containment bag is in a deployed configuration, the containment bag and open end are sized to allow the diaper member to pass through the open end and be contained in the interior portion;
a first containment slot integral with the diaper member at an upper portion on a rear exterior of the diaper member, wherein the exterior surface of the diaper member is continuous in and around the first containment slot;
a first slot cover integral with the diaper member and configured to be removably positioned over the first containment slot; and
wherein when the containment bag is in a storage configuration and the first slot cover positioned over the first containment slot, the containment bag is sized to be enclosed in the first containment slot beneath the first slot cover.

12. The diaper assembly with a deployable containment bag of claim 11, wherein the first containment slot is defined by a depression in the exterior surface of the diaper member.

13. The diaper assembly with a deployable containment bag of claim 11, wherein first containment slot is elongated and extends laterally across an upper portion on a rear exterior of the diaper member.

14. The diaper assembly with a deployable containment bag of claim 11, wherein the first slot cover is configured to be removably attached to the exterior surface of the diaper member adjacent to the first containment slot and when attached to the exterior surface of the diaper member adjacent to the first containment slot, is sized to extend over the first containment slot.

15. The diaper assembly with a deployable containment bag of claim 14, wherein the first slot cover is configured to be removably attached to the exterior surface of the diaper member with an adhesive coating.

16. The diaper assembly with a deployable containment bag of claim 11, wherein the containment bag integral with the first slot cover such that when the first slot cover is removed from a position over the first containment slot, the containment bag is automatically removed from the first containment slot.

17. The diaper assembly with a deployable containment bag of claim 11, wherein when the containment bag is in the deployed configuration, the containment bag is structured such that the open end can be sealed and cause the interior portion becoming an airtight enclosure.

18. The diaper assembly with a deployable containment bag of claim 11, additionally comprising a second containment slot integral with the diaper member at an upper portion on a front exterior of the diaper member and a second slot cover integral with the diaper member and configured to be removably positioned over the second containment slot.

19. A diaper assembly with a deployable containment bag, comprising:
a diaper member configured to be worn by a user and at least one of absorb and contain urine and feces to prevent soiling of clothing that is also being worn by the user, wherein the diaper member includes an exterior surface;
a containment bag defined by a non-rigid container having an open end and an interior portion, wherein when the containment bag is in a deployed configuration, the containment bag and open end are sized to allow the diaper member to pass through the open end and be contained in the interior portion;
an elongated first containment slot which extends laterally across the diaper member at an upper portion on a rear exterior of the diaper member, wherein the exterior surface of the diaper member is continuous in and around the first containment slot;
wherein the first containment slot is defined by a depression in the exterior surface of the diaper member;
a first slot cover configured to be removably attached to the exterior surface of the diaper member adjacent to the first containment slot, and when attached to the exterior surface of the diaper member adjacent to the first containment slot, sized to extend over the first containment slot;
wherein when the containment bag is in a storage configuration and the first slot cover positioned over the first containment slot, the containment bag is sized to be enclosed in the first containment slot beneath the first slot cover;
wherein when the containment bag is in the deployed configuration, the containment bag is structured such that the open end can be sealed and cause the interior portion becoming an airtight enclosure.

20. The diaper assembly with a deployable containment bag of claim 19, additionally comprising a second containment slot integral with the diaper member at an upper portion on a front exterior of the diaper member and a second slot cover integral with the diaper member and configured to be removably positioned over the second containment slot.
